(19) United States
(12) Patent Application Publication
(10) Pub. No.: US 2025/0193985 A1
(43) Pub. Date: Jun. 12, 2025

(54) INFORMATION PROCESSING METHOD AND INFORMATION PROCESSING SYSTEM

(71) Applicant: Panasonic Holdings Corporation, Osaka (JP)

(72) Inventors: HITOSHI TOMINAGA, Kyoto (JP); HIROTO YANAGAWA, Osaka (JP); NAOMI SATOI, Osaka (JP); YUMIKO OHNO, Osaka (JP); YUKIKO UCHIDA, Kyoto (JP); MASATAKA NAKAYAMA, Kyoto (JP)

(21) Appl. No.: 19/054,808

(22) Filed: Feb. 15, 2025

Related U.S. Application Data
(63) Continuation of application No. PCT/JP2023/027990, filed on Jul. 31, 2023.

(30) Foreign Application Priority Data
Aug. 31, 2022 (JP) 2022-137694

(51) Int. Cl. H05B 47/105; A61B 5/026; A61B 5/1455; A61B 5/16

(52) U.S. Cl. CPC H05B 47/105; A61B 5/0261; A61B 5/1455; A61B 5/165

(57) ABSTRACT

An information processing method includes acquiring first cerebral blood flow information corresponding to a temporal change of cerebral blood flow in a central portion of a user's forehead in a left-right direction by using a sensor; acquiring second cerebral blood flow information corresponding to a temporal change of cerebral blood flow in an end portion of the user's forehead in the left-right direction by using the sensor; and determining by a processor whether a user's level of divergent thinking is rising or a user's level of convergent thinking is rising on the basis of comparison between the first cerebral blood flow information and the second cerebral blood flow information.

### BACKGROUND

### 1. Technical Field

The present disclosure relates to an information processing method and an information processing system.

### 2. Description of the Related Art

There are two kinds of human creative thinking: convergent thinking of logically deducing a single solution from known information and divergent thinking of producing new ideas from known information.

Conventionally, a technique of estimating a person's level of concentration on the basis of cerebral blood flow is known (see Japanese Unexamined Patent Application Publication No. 2017-140107).

### SUMMARY

One non-limiting and exemplary embodiment provides an information processing method and others that make it possible to objectively evaluate a subject's creative thinking state.

In one general aspect, the techniques disclosed here feature an information processing method including acquiring first cerebral blood flow information corresponding to a temporal change of cerebral blood flow in a central portion of a user's forehead in a left-right direction by using a sensor; acquiring second cerebral blood flow information corresponding to a temporal change of cerebral blood flow in an end portion of the user's forehead in the left-right direction by using the sensor; and determining by a processor whether a user's level of divergent thinking is rising or a user's level of convergent thinking is rising on the basis of comparison between the first cerebral blood flow information and the second cerebral blood flow information.

The information processing method according to the present disclosure makes it possible to objectively evaluate a subject's creative thinking state.

It should be noted that general or specific embodiments may be implemented as a system, a device, an integrated circuit, a computer program, a computer-readable storage medium such as a CD-ROM, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a schematic view illustrating appearance of a cerebral blood flow measurement device;

FIG. 1B is an explanatory view schematically illustrating a configuration of the cerebral blood flow measurement device;

FIG. 1C is an explanatory view illustrating subjects' cerebral blood flow change amounts for each thinking state;

FIG. 2A is a first explanatory view illustrating a relationship between subject's cerebral blood flow change amount and thinking state;

FIG. 2B is a second explanatory view illustrating a relationship between subject's cerebral blood flow change amount and thinking state;

FIG. 3 is a block diagram illustrating an example of a configuration of a bioinstrumentation device according to an embodiment;

FIG. 4A is a first flowchart illustrating a method for determining a user's thinking state performed by the bioinstrumentation device according to the embodiment;

FIG. 4B is a second flowchart illustrating a method for determining a user's thinking state performed by the bioinstrumentation device according to the embodiment;

FIG. 5 is an explanatory view illustrating an example of a user's thinking state determined by the bioinstrumentation device according to the embodiment;

FIG. 6A is an explanatory view illustrating a first example of presentation of a result of determination performed by the bioinstrumentation device according to the embodiment;

FIG. 6B is an explanatory view illustrating a second example of display of the determination result according to the embodiment;

FIG. 6C is an explanatory view illustrating a third example of display of the determination result according to the embodiment;

FIG. 6D is an explanatory view illustrating a fourth example of display of the determination result according to the embodiment;

FIG. 6E is an explanatory view illustrating a fifth example of display of the determination result according to the embodiment;

FIG. 6F is an explanatory view illustrating a sixth example of display of the determination result according to the embodiment;

FIG. 6G is an explanatory view illustrating a seventh example of display of the determination result according to the embodiment;

FIG. 7 is a flowchart illustrating an example of processing for controlling lighting in accordance with a determination result according to the embodiment; and

FIG. 8 is an explanatory view illustrating a relationship between illuminance and a color temperature of lighting in lighting control and a user's thinking state according to the embodiment.

### DETAILED DESCRIPTIONS

### Underlying Knowledge Forming Basis of the Present Disclosure

Knowledge found by the inventors of the present invention is described before describing an embodiment of the present disclosure.

Against the background of a labor shortage resulting from the falling birth rate and the aging population, the government is promoting work style reforms. Companies are tackling creating various systems for improving employees' intellectual productivity. Employees' motivation to work and office spaces are closely related, and development of environment design or space control for improving intellectual productivity is under way.

For improvement of intellectual productivity, not only whether or not employees are able to concentrate on work, but also whether or not employees are able to do more creative thinking matter. There are two kinds of human creative thinking: convergent thinking of logically deducing a single solution from known information and divergent thinking of producing new ideas from known information.

Conventionally, studies on methods for evaluating effects of a surrounding environment on a thinking state have been conducted, but no standardized objective and quantitative index has been established.

The present disclosure provides an information processing method and others that make it possible to objectively evaluate a human creating thinking state.

An example of biological information of a subject doing a task that requires convergent thinking and biological information of a subject doing a task that requires divergent thinking is described below. The biological information is information concerning cerebral blood flow (more specifically, a cerebral blood flow change amount in a forehead central portion and a cerebral blood flow change amount in a forehead end portion).

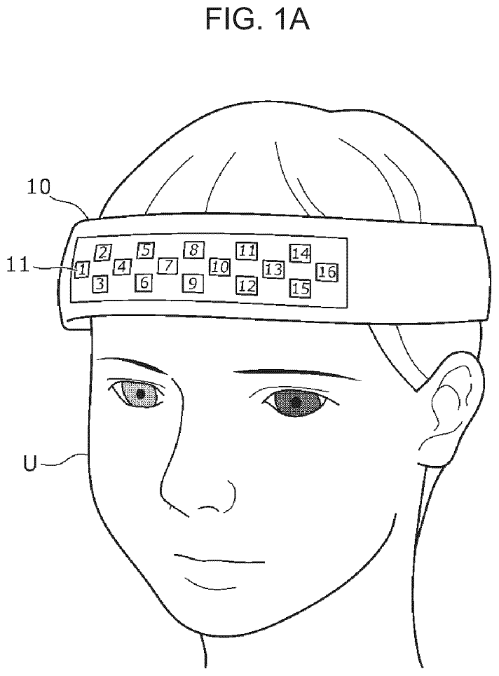

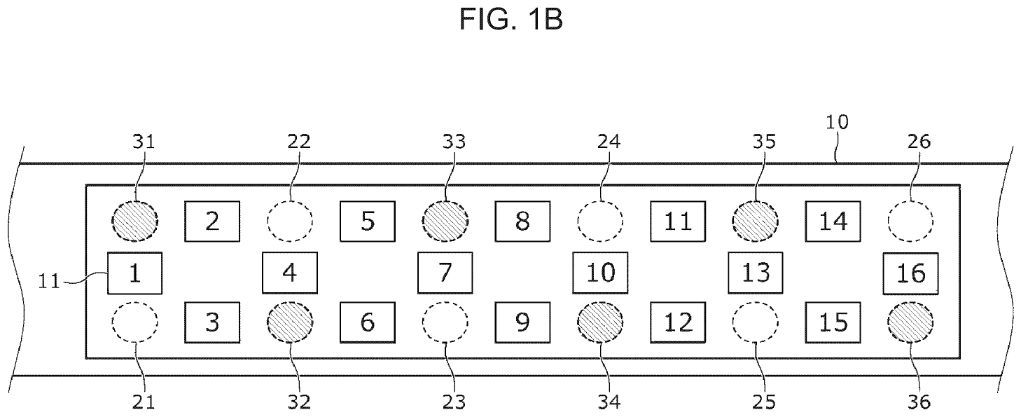

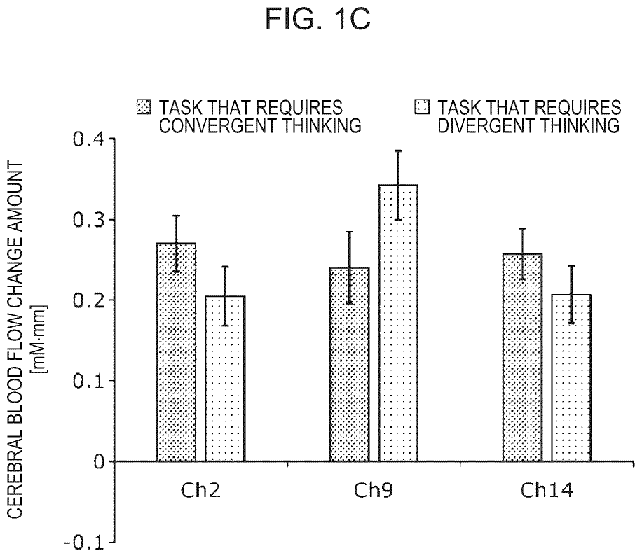

FIG. 1A is a schematic view illustrating appearance of a cerebral blood flow measurement device 10. FIG. 1B is an explanatory view schematically illustrating a configuration of the cerebral blood flow measurement device 10. FIG. 1C is an explanatory view illustrating subjects' cerebral blood flow change amounts for each thinking state. A relationship between subject's cerebral blood flow amount and thinking state is described while referring to FIGS. 1A to 1C.

The cerebral blood flow measurement device 10 is mounted over a forehead of a user U, who is a subject, as illustrated in FIG. 1A.

The cerebral blood flow measurement device 10 can measure a cerebral blood flow amount (more specifically, an amount of oxygenated hemoglobin OxyHb) of the user U at positions (also referred to as measurement positions) 11 in a region covering a central portion (also referred to as a “forehead central portion”) of the forehead of the user U in a left-right direction and an end portion (also referred to as a “forehead end portion”) of the forehead in the left-right direction. The number of measurement positions 11 is, for example, 16 and are given characters “1” to “16” in FIG. 1A. The measurement positions 11 are positioned to face positions where a cerebral blood flow amount is to be measured on the forehead of the user U when the cerebral blood flow measurement device 10 is mounted on the forehead.

The cerebral blood flow measurement device 10 includes one or more light emitting units and one or more light receiving units that measure cerebral blood flow amounts at the measurement positions 11.

An example of layout of the one or more light emitting units and the one or more light receiving units is described with reference to FIG. 1B.

In FIG. 1B, the 16 measurement positions 11, 6 light emitting units 21, 22, 23, 24, 25, and 26 (also referred to as the light emitting units 21 and others), and 6 light receiving units 31, 32, 33, 34, 35, and 36 (also referred to as the light receiving units 31 and others) of the cerebral blood flow measurement device 10 are illustrated.

The light emitting units 21 and others are provided on a surface facing the forehead of the user U, that is, a surface that can emit light toward the forehead of the user U when the cerebral blood flow measurement device 10 is mounted on the forehead of the user U. The light receiving units 31 and others are provided on the surface facing the forehead of the user U, that is, a surface that can receive light from the forehead of the user U when the cerebral blood flow measurement device 10 is mounted on the forehead of the user U.

The cerebral blood flow measurement device 10 measures a cerebral blood flow amount of the forehead of the user U by using a light emitting unit located relatively close to a position (also referred to as a target position) where the cerebral blood flow amount is to be measured among the 6 light emitting units 21 and others and a light receiving unit located relatively close to the target position among the 6 light receiving units 31 and others.

For example, in a case where a cerebral blood flow amount at a measurement position 11 indicated by “2” is to be measured, the light emitting unit 22 relatively close to the measurement position 11 indicated by “2” and the light receiving unit 31 relatively close to the measurement position 11 indicated by “2” are used.

Similarly, for example, in a case where a cerebral blood flow amount at a measurement position 11 indicated by “9” is to be measured, the light emitting unit 23 relatively close to the measurement position 11 indicated by “9” and the light receiving unit 34 relatively close to the measurement position 11 indicated by “9” are used.

Note that the layout of the light emitting units 21 and others and the light receiving units 31 and others, the number of light emitting units, and the number of light receiving units illustrated in FIG. 1B are examples and are not restrictive.

Measurement values of cerebral blood flow, that is, cerebral blood flow amounts at the measurement positions 11 are also referred to as channels. A channel given a character given to a measurement position 11 indicates a cerebral blood flow amount at this measurement position 11.

For example, channels 1 to 6 indicate cerebral blood flow amounts in a forehead end portion (on the right) of the user U, channels 7 to 10 indicate cerebral blood flow amounts in a forehead central portion of the user U, and channels 11 to 16 indicate cerebral blood flow amounts in a forehead end portion (on the left) of the user U.

FIG. 1C illustrates an example of cerebral blood flow change amounts measured by the cerebral blood flow measurement device 10 in a case where 50 subjects do a task requiring convergent thinking and a task requiring divergent thinking. In FIG. 1C, an average of cerebral blood flow change amounts of the 50 subjects is indicated by a bar graph, and a standard error of the cerebral blood flow change amounts of the 50 subjects is indicated by an error bar.

The cerebral blood flow change amounts of the channels 2, 9, and 14 illustrated in FIG. 1C are values obtained from cerebral blood flow amounts of the channels 2, 9, and 14, and are change amounts of cerebral blood flow amounts from the start of the tasks to the end of the tasks. A period from the start to the end of the tasks is approximately 5 minutes. The cerebral blood flow change amounts of the channels 2, 9, and 14 illustrated in FIG. 1C indicate cerebral blood flow change amounts in the forehead end portion (on the right), cerebral blood flow change amounts in the forehead central portion, and cerebral blood flow change amounts in the forehead end portion (on the left) of the subjects, respectively.

As is clear from the cerebral blood flow change amounts illustrated in FIG. 1C, cerebral blood flow change amounts in the forehead central portion and the forehead end portions are larger than 0 in a case where the subjects are performing the task that requires convergent thinking and the task that requires divergent thinking. It is also clear that in a case where the subjects are performing the task that requires divergent thinking, the cerebral blood flow change amount in the forehead central portion is larger than the cerebral blood flow change amounts in the forehead end portions. It is also clear that there is no significant difference between the forehead end portion on the left and the forehead end portion on the right.

As is clear from the cerebral blood flow change amounts illustrated in FIG. 1C, for example, there is a possibility that it can be determined that a subject's level of convergent thinking or divergent thinking is rising in a case where cerebral blood flow change amounts in a state where the subject is performing a task (also referred to as a performing state) from cerebral blood flow amounts in a state where the subject is not performing a task (also referred to as a resting state) are larger than 0. Furthermore, there is a possibility that it can be determined that a subject's level of divergent thinking is rising in a case where a cerebral blood flow change amount in the forehead central portion is larger than cerebral blood flow change amounts in the forehead end portions. Furthermore, there is a possibility that it can be determined that a subject's level of convergent thinking is rising in a case where change amounts of cerebral blood flow amounts in the forehead end portions are larger than a change amount of a cerebral blood flow amount in the forehead central portion.

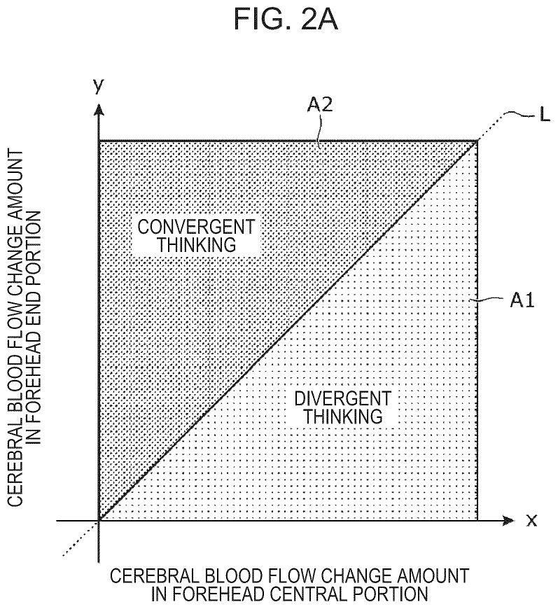

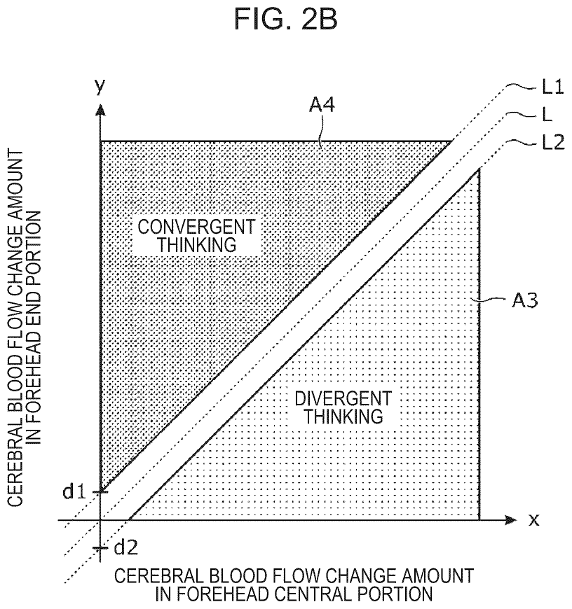

FIG. 2A is a first explanatory view illustrating a relationship between subject's cerebral blood flow change amount and thinking state.

In FIG. 2A, the x-axis represents a cerebral blood flow change amount in a forehead central portion, and the y-axis represents a cerebral blood flow change amount in a forehead end portion, and a subject's thinking state determined from the cerebral blood flow change amount in the forehead central portion and the cerebral blood flow change amount in the forehead end portion is illustrated.

In FIG. 2A, a region where x>0 and y>0, that is, the first quadrant corresponds to a state where both of the cerebral blood flow change amount in the forehead central portion and the cerebral blood flow change amount in the forehead end portion are increasing. Measured values of the cerebral blood flow change amount in the forehead central portion and the cerebral blood flow change amount in the forehead end portion of the subject correspond to one position in the first quadrant.

There is a possibility that whether or not a subject's level of convergent thinking is rising or whether or not a subject's level of divergent thinking is rising can be determined on the basis of a positional relationship between the measured values of the cerebral blood flow change amount in the forehead central portion and the cerebral blood flow change amount in the forehead end portion of the subject (i.e., one position in the first quadrant) and a straight line L (corresponding to y=x).

Specifically, there is a possibility that it can be determined that a subject's level of divergent thinking is rising in a case where the cerebral blood flow change amount in the forehead end portion of the subject is smaller than the cerebral blood flow change amount in the forehead central portion of the subject (in other words, in a case where y<x, that is, in a case of a point located in a lower right region A1 relative to the straight line L).

On the other hand, there is a possibility that it can be determined that a subject's level of convergent thinking is rising in a case where the cerebral blood flow change amount in the forehead end portion of the subject is larger than the cerebral blood flow change amount in the forehead central portion of the subject (in other words, in a case where y>x, that is, in a case of a point located in an upper left region A2 relative to the straight line L).

Note that it may be regarded that there is a possibility that it can be determined that a subject's level of divergent thinking is rising or it may be regarded that there is a possibility that it can be determined that a subject's level of convergent thinking is rising in a case where the cerebral blood flow change amount in the forehead end portion of the subject is equal to the cerebral blood flow change amount in the forehead central portion of the subject (in other words, in a case where y=x, that is. in a case of a point located on the straight line L).

Note that the rise in level of convergent thinking may mean that the subject is in a divergent thinking state and is changing toward a convergent thinking state or may mean that the subject is in a convergent thinking state and a level of the convergent thinking is rising. Similarly, the rise in level of divergent thinking may mean that the subject is in a convergent thinking state and is changing toward a divergent thinking state or may mean that the subject is in a divergent thinking state and a level of the divergent thinking is rising.

Note that a buffer region may be provided to improve accuracy of determination of a thinking state.

FIG. 2B is a second explanatory view illustrating a relationship between subject's cerebral blood flow change amount and thinking state.

In FIG. 2B, the x-axis represents a cerebral blood flow change amount in a forehead central portion, and the y-axis represents a cerebral blood flow change amount in a forehead end portion, and a subject's thinking state determined from the cerebral blood flow change amount in the forehead central portion and the cerebral blood flow change amount in the forehead end portion is illustrated, as in FIG. 2A.

In FIG. 2B, a straight line L1 shifted from the straight line L by d1 in a y-axis positive direction and a straight line L2 shifted from the straight line L by d2 in a y-axis negative direction are provided.

There is a possibility that whether or not a subject's level of convergent thinking is rising or whether or not a subject's level of divergent thinking is rising can be determined on the basis of a positional relationship between measured values of the cerebral blood flow change amount in the forehead central portion and the cerebral blood flow change amount in the forehead end portion of the subject (i.e., one position in the first quadrant) and the straight lines L1 and L2.

Specifically, there is a possibility that the subject's level of divergent thinking is rising in a case where the cerebral blood flow change amount in the forehead end portion of the subject is smaller than the cerebral blood flow change amount in the forehead central portion of the subject by d2 or more (in other words, in a case of y<x−d2, that is, in a case of a point located in a lower right region A3 relative to the straight line L2).

On the other hand, there is a possibility that the subject's level of convergent thinking is rising in a case where the cerebral blood flow change amount in the forehead end portion of the subject is larger than the cerebral blood flow change amount in the forehead central portion of the subject by d1 or more (in other words, in a case of y>x+d1, that is, in a case of a point located in an upper left region A4 relative to the straight line L1).

Note that there is a possibility that it can be determined that neither the subject's level of convergent thinking nor the subject's level of divergent thinking is rising in a case where the cerebral blood flow change amount in the forehead end portion of the subject is larger than a value that is smaller than the cerebral blood flow change amount in the forehead central portion of the subject by d2 and is smaller than a value that is larger than the cerebral blood flow change amount in the forehead central portion of the subject by d1 (in other words, in a case where x−d2<y<x+d1, that is, in a case of a point located between the straight line L1 and the straight line L2).

Note that it may be regarded that there is a possibility that it can be determined that the subject's level of divergent thinking is rising or it may be regarded that there is a possibility that it can be determined that the subject's level of convergent thinking is rising in a case where the cerebral blood flow change amount in the forehead end portion of the subject is smaller than the cerebral blood flow change amount in the forehead central portion of the subject by d2 (in other words, in a case where y=x−d2, that is, in a case of a point located on the straight line L2) or in a case where the cerebral blood flow change amount in the forehead end portion of the subject is larger than the cerebral blood flow change amount in the forehead central portion of the subject by d1 (in other words, in a case where y=x+d1, that is, in a case of a point located on the straight line L1).

Note that a subject's thinking state may be determined in a case where measured values remain in the same region for more than a preset period t.

The following illustrates a technique obtained from the disclosure of the present specification and describes effects and the like obtained from the technique.
(1) An information processing method including: acquiring first cerebral blood flow information corresponding to a temporal change of cerebral blood flow in a central portion of a user's forehead in a left-right direction by using a sensor; acquiring second cerebral blood flow information corresponding to a temporal change of cerebral blood flow in an end portion of the user's forehead in the left-right direction by using the sensor; and determining by a processor whether a user's level of divergent thinking is rising or a user's level of convergent thinking is rising on the basis of comparison between the first cerebral blood flow information and the second cerebral blood flow information.

According to the above aspect, whether or not a user's level of divergent thinking is rising or whether or not a user's level of convergent thinking is rising can be determined by using the temporal changes of the cerebral blood flow in the central portion and the end portion of the user's forehead. Conventionally, no technique of determining a user's level of divergent thinking or user's level of convergent thinking from temporal changes of cerebral blood flow in a central portion and an end portion of a user's forehead is known. According to the above information processing method, whether or not a user's level of divergent thinking is rising or whether or not a user's level of convergent thinking is rising can be determined by using the temporal changes of the cerebral blood flow in the central portion and the end portion of the user's forehead. According to the above information processing method, it is thus possible to objectively evaluate a subject's creative thinking state.
(2) The information processing method according to (1), in which the sensor acquires a cerebral blood flow amount in the central portion at a first time point and a cerebral blood flow amount in the central portion at a second time point later than the first time point and acquires a cerebral blood flow amount in the end portion at the first time point and a cerebral blood flow amount in the end portion at the second time point, and the determining includes determining that the user's level of divergent thinking is rising in a case where it is determined that an increase amount of the cerebral blood flow amount in the central portion at the second time point from the cerebral blood flow amount in the central portion at the first time point is larger than an increase amount of the cerebral blood flow amount in the end portion at the second time point from the cerebral blood flow amount in the end portion at the first time point.

According to the above aspect, it is possible to more easily determine that a user's level of divergent thinking is rising by using increase amounts of cerebral blood flow amounts in the central portion and the end portion of the user's forehead from the first time point to the second time point. According to the above information processing method, it is thus possible to more easily objectively evaluate a subject's creative thinking state.
(3) The information processing method according to (1) or (2), in which the sensor repeatedly acquires a cerebral blood flow amount in the central portion and repeatedly acquires a cerebral blood flow amount in the end portion, and the determining includes determining that the user's level of divergent thinking is rising in a case where it is determined that a rate of change of the cerebral blood flow amount in the central portion per time is higher than a rate of change of the cerebral blood flow amount in the end portion per time.

According to the above aspect, it is possible to more easily determine that a user's level of divergent thinking is rising by using rates of change of cerebral blood flow amounts in the central portion and the end portion of the user's forehead per time from the first time point to the second time point. This produces another effect that it can be determined that a user's level of divergent thinking is rising only by measurement of cerebral blood flow amounts in a relatively short period since a rate of change of cerebral blood flow per time (in other words, a slope of a graph showing a temporal change of cerebral blood flow) is used. According to the above information processing method, it is thus possible to more easily objectively evaluate a subject's creative thinking state in a short period. Since a subject's creative thinking state can be evaluated in a short time, a processing amount of a processor can be reduced.
(4) The information processing method according to any one of (1) to (3), in which the sensor acquires a cerebral blood flow amount in the central portion at a first time point and a cerebral blood flow amount in the central portion at a second time point later than the first time point and acquires a cerebral blood flow amount in the end portion at the first time point and a cerebral blood flow amount in the end portion at the second time point, and the determining includes determining that the user's level of convergent thinking is rising in a case where it is determined that an increase amount of the cerebral blood flow amount in the end portion at the second time point from the cerebral blood flow amount in the end portion at the first time point is larger than an increase amount of the cerebral blood flow amount in the central portion at the second time point from the cerebral blood flow amount in the central portion at the first time point.

According to the above aspect, it is possible to more easily determine that a user's level of convergent thinking is rising by using increase amounts of cerebral blood flow amounts in the central portion and the end portion of the user's forehead from the first time point to the second time point. According to the above information processing method, it is thus possible to more easily objectively evaluate a subject's creative thinking state.
(5) The information processing method according to any one of (1) to (4), in which the sensor acquires the first cerebral blood flow information and the second cerebral blood flow information in each of periods, the determining includes determining, for each of the periods, whether a user's level of divergent thinking in the period is rising or a user's level of convergent thinking in the period is rising on the basis of the first cerebral blood flow information and the second cerebral blood flow information acquired in the period, and the information processing method further includes generating a graph showing proportions of a period where the user's level of convergent thinking is rising and a period where the user's level of divergent thinking is rising among the periods and displaying the graph.

According to the above aspect, a graph showing a proportion of the user's thinking state in each of periods is displayed. This contributes to allowing a viewer of the graph to collectively grasp the user's thinking states over the periods. Therefore, according to the information processing method, it is possible to objectively evaluate a subject's creative thinking state over periods and allow a viewer to easily grasp the thinking state.
(6) The information processing method according to (2) or (3), further including controlling a lighting device that illuminates an area around the user to raise a color temperature of illuminating light and raise illuminance of the illuminating light.

According to the above aspect, the user can be properly guided to a convergent thinking state by using the lighting device in a case where it is determined that a user's level of divergent thinking is rising. Therefore, according to the information processing method, it is possible to objectively evaluate a subject's creative thinking state and guide a user determined to have a rising level of divergent thinking to a convergent thinking state.
(7) The information processing method according to (4), further including controlling a lighting device that illuminates an area around the user to lower a color temperature of illuminating light and raise illuminance of the illuminating light.

According to the above aspect, the user can be properly guided to a divergent thinking state by using the lighting device in a case where it is determined that a user's level of convergent thinking is rising. Therefore, according to the information processing method, it is possible to objectively evaluate a subject's creative thinking state and guide a user determined to have a rising level of convergent thinking to a divergent thinking state.
(8) An information processing method including causing a processor to: acquire target information indicative of a target state which a user aims for; acquire cerebral blood flow information indicative of a temporal change of cerebral blood flow of the user; determine a thinking state of the user on the basis of the cerebral blood flow information; compare the target state indicated by the target information and the determined thinking state of the user; and perform control of changing a property of illuminating light emitted by a lighting device that illuminates an area around the user in a case where the target state and the determined thinking state of the user do not match as a result of the comparison.

According to the above aspect, effects similar to those of the above information processing method are produced.

According to the above aspect, the user's thinking state can be properly guided to the target state by using the lighting device in a case where the user's thinking state determined from the user's cerebral blood flow information is different from the target state. Therefore, according to the information processing method, it is possible to objectively evaluate a subject's creative thinking state and guide the user's thinking state to the target state.
(9) An information processing system including: a light source that emits first light to a central portion of a user's forehead in a left-right direction and emits second light to an end portion of the user's forehead in the left-right direction; a detector that detects third light originating from the first light and coming from the central portion and fourth light originating from the second light and coming from the end portion; a processing circuit; and a display device, in which the processing circuit generates first cerebral blood flow information including information on a temporal change of a hemoglobin concentration of cerebral blood flow in the central portion on the basis of the third light, generates second cerebral blood flow information including information on a temporal change of a hemoglobin concentration of cerebral blood flow in the end portion on the basis of the fourth light, performs comparison processing of comparing a change amount of the hemoglobin concentration of the cerebral blood flow in the central portion and a change amount of the hemoglobin concentration of the cerebral blood flow in the end portion on the basis of the first cerebral blood flow information and the second cerebral blood flow information, performs determination processing of determining that a user's level of divergent thinking is rising in a case where it is determined on the basis of the comparison processing that the change amount of the hemoglobin concentration of the cerebral blood flow in the central portion is larger than the change amount of the hemoglobin concentration of the cerebral blood flow in the end portion, and determining that a user's level of convergent thinking is rising in a case where it is determined on the basis of the comparison processing that the change amount of the hemoglobin concentration of the cerebral blood flow in the end portion is larger than the change amount of the hemoglobin concentration of the cerebral blood flow in the central portion, and displays a result of the determination processing on a display.

According to the above aspect, effects similar to those of the above information processing method are produced.

Note that these general or specific aspects may be implemented as a system, a device, an integrated circuit, a computer program, a computer-readable recording medium such as a CD-ROM, or any combination thereof.

An embodiment is specifically described below with reference to the drawings.

Note that the embodiment described below illustrates a general or specific example. Numerical values, shapes, materials, constituent elements, positions of the constituent elements, ways in which the constituent elements are connected, steps, an order of steps, and the like in the embodiment below are examples and do not limit the present disclosure. Among constituent elements in the embodiment below, constituent elements that are not described in independent claims indicating highest concepts are described as optional constituent elements.

### Embodiment

In the present embodiment, an information processing method and others that make it possible to objectively evaluate a subject's creative thinking state are described.

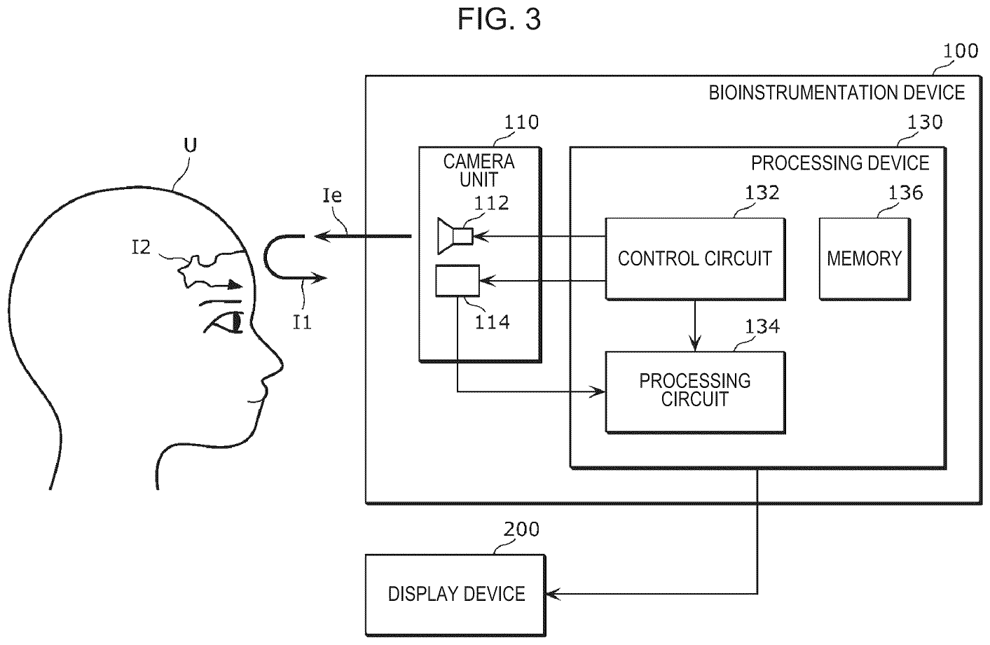

FIG. 3 is a block diagram illustrating an example of a configuration of a bioinstrumentation device 100 according to the present embodiment.

The bioinstrumentation device 100 is an information processing device that measures biological information of a user U. The bioinstrumentation device 100 can be a non-contact type near-infrared spectroscopy (NIRS) device. The biological information of the user U is information concerning cerebral blood flow of the user U (more specifically, a cerebral blood flow change amount in a forehead central portion and a cerebral blood flow change amount in a forehead end portion). It can be said that the bioinstrumentation device 100 is a sensor that measures information concerning cerebral blood flow of the user U.

The bioinstrumentation device 100 includes a camera unit 110, a processing device 130, and a display device 200.

The camera unit 110 measures a cerebral blood flow amount in a forehead central portion of the user U and a cerebral blood flow amount in a forehead end portion of the user U. The camera unit 110 includes one or more light emitting units 112 and one or more light receiving units 114. The one or more light emitting units 112 and the one or more light receiving units 114 correspond, for example, to the light emitting units 21 and others and the light receiving units 31 and others illustrated in FIG. 1B, respectively.

The light emitting unit 112 is a near-infrared light source. Near-infrared light emitted by the light emitting unit 112 includes near-infrared light of a wavelength (e.g., a wavelength of 800 nm) that is easily absorbed in hemoglobin.

More specifically, the light emitting unit 112 included in the camera unit 110 that measures a cerebral blood flow amount in a forehead central portion of the user U emits near-infrared light (corresponding to first light) to the forehead central portion of the user U. The light emitting unit 112 included in the camera unit 110 that measures a cerebral blood flow amount in the forehead end portion of the user U emits near-infrared light (corresponding to second light) to the forehead end portion of the user U.

The light receiving unit 114 is a sensor (also called a detector) that detects reflected light of the near-infrared light emitted by the light emitting unit 112 and is, for example, an image sensor. More specifically, the light receiving unit 114 (corresponding to a first sensor) included in the camera unit 110 that measures a cerebral blood flow amount in the forehead central portion of the user U detects near-infrared light (corresponding to third light) originating from the first light and coming from the forehead central portion. The light receiving unit 114 (corresponding to a second sensor) included in the camera unit 110 that measures a cerebral blood flow amount in the forehead end portion of the user U detects near-infrared light (corresponding to fourth light) originating from the second light and coming from the forehead end portion.

Reflected light from a whole forehead including a forehead end portion, a central portion, and a portion different from the forehead end portion and the central portion may be detected, and a cerebral blood flow amount in the forehead end portion and a cerebral blood flow amount in the central portion may be derived on the basis of the reflected light without deriving a cerebral blood flow amount in the portion different from the forehead end portion and the central portion. Since reflected light from the forehead end portion and the central portion is detected and reflected light from the portion different from the forehead end portion and the central portion need not be detected, unnecessary data acquisition and processing can be reduced while keeping accuracy of measurement. This makes it possible to reduce a processing load.

The camera unit 110 irradiates the forehead portion of the user U with near-infrared light le by using the light emitting unit 112 under control of a control circuit 132. The near-infrared light Ie is separated into a surface component 11, which is a component reflected on a surface of the forehead portion, and a diffuse component 12, which passes through scalp and skull and is diffused by cerebral neocortex. A portion of the diffuse component 12 returns to the camera unit 110 via the cerebral neocortex of the user U and is then received by the light receiving unit 114. A remaining portion of the diffuse component 12 excluding this portion is absorbed by hemoglobin contained in cerebral blood flow or is diffused in the cerebral neocortex. Accordingly, a temporal change of the cerebral blood flow is reflected in an intensity of light received by the light receiving unit 114.

The light receiving unit 114 generates information indicative of the intensity of the received light and supplies the information to the processing device 130 (more specifically, a processing circuit 134).

The processing device 130 includes the control circuit 132, the processing circuit 134, and a memory 136.

The control circuit 132 controls the camera unit 110. Specifically, the control circuit 132 controls light emission of the light emitting unit 112 and light reception of the light receiving unit 114. The control circuit 132 can be a processor such as a central processing unit (CPU) or an integrated circuit such as a microcomputer including a processor.

The control circuit 132 controls the light emitting unit 112 to emit light at a timing at which a cerebral blood flow amount of the user U should be measured and controls the light receiving unit 114 to receive light (i.e., the surface component 11, the above portion of the diffuse component 12, and the like) originating from the light emitted by the light emitting unit 112 and arriving from the user U. The control circuit 132 continuously acquires an intensity of near-infrared light reflected from the user U by using the camera unit 110 during a period from a time at which the user U is in a resting state to a time at which a thinking state of the user U should be determined.

The processing circuit 134 acquires a signal indicative of an intensity of near-infrared light received by the light receiving unit 114 from the camera unit 110 and generates cerebral blood flow information of the user U by performing arithmetic processing on the signal. The processing circuit 134 determines the thinking state of the user U on the basis of the cerebral blood flow information of the user U.

The cerebral blood flow information generated by the processing circuit 134 includes first cerebral blood flow information and second cerebral blood flow information. It can also be said that the first cerebral blood flow information and the second cerebral blood flow information are acquired by the bioinstrumentation device 100, that is, a sensor.

The first cerebral blood flow information is cerebral blood flow information acquired by using the camera unit 110 (more specifically, the light receiving unit 114) that measures a cerebral blood flow amount in the forehead central portion of the user U. In other words, the first cerebral blood flow information is acquired as a result of arithmetic processing performed by the processing circuit 134 on a signal acquired by the camera unit 110 that measures the cerebral blood flow amount in the forehead central portion of the user U. The first cerebral blood flow information includes information on a temporal change of a hemoglobin concentration of cerebral blood flow in the forehead central portion.

The second cerebral blood flow information is cerebral blood flow information acquired by using the camera unit 110 (more specifically, the light receiving unit 114) that measures a cerebral blood flow amount in the forehead end portion of the user U. In other words, the second cerebral blood flow information is acquired as a result of arithmetic processing performed by the processing circuit 134 on a signal acquired by the camera unit 110 that measures the cerebral blood flow amount in the forehead end portion of the user U. The second cerebral blood flow information includes information on a temporal change of a hemoglobin concentration of cerebral blood flow in the forehead end portion.

As determination of the thinking state of the user U, the processing circuit 134 compares the first cerebral blood flow information and the second cerebral blood flow information and determines whether a user U's level of divergent thinking is rising or whether a user U's level of convergent thinking is rising on the basis of a result of the comparison. More specifically, the processing circuit 134 determines that the user U's level of divergent thinking is rising in a case where it is determined that a change amount of a hemoglobin concentration of cerebral blood flow in the forehead central portion is larger than a change amount of a hemoglobin concentration of cerebral blood flow in the forehead end portion and determines that the user U's level of convergent thinking is rising in a case where the change amount of the hemoglobin concentration of the cerebral blood flow in the forehead end portion is larger than the change amount of the hemoglobin concentration of the cerebral blood flow in the forehead central portion.

The memory 136 is a storage device used as a work area used during operation of the control circuit 132. The memory 136 is also used as a storage region for a measured value of a cerebral blood flow amount.

The display device 200 displays a result of measurement performed by the bioinstrumentation device 100. More specifically, the display device 200 displays a result of determination of the thinking state of the user U performed by the processing circuit 134. The display device 200 can be a smartphone, a tablet PC, a personal computer, or the like connected to the bioinstrumentation device 100. Connection between the display device 200 and the bioinstrumentation device 100 may be wireless communication such as Wi-Fi (Registered Trademark) or Bluetooth (Registered Trademark) or may be wired communication such a USB or Ethernet (Registered Trademark).

Note that another output device (e.g., a speaker or a communication interface) may be connected to the bioinstrumentation device 100 instead of the display device 200 or together with the display device 200. In a case where a speaker is connected to the bioinstrumentation device 100, the bioinstrumentation device 100 generates audio data indicative of a determination result, supplies the audio data to the speaker, and causes the speaker to output sound.

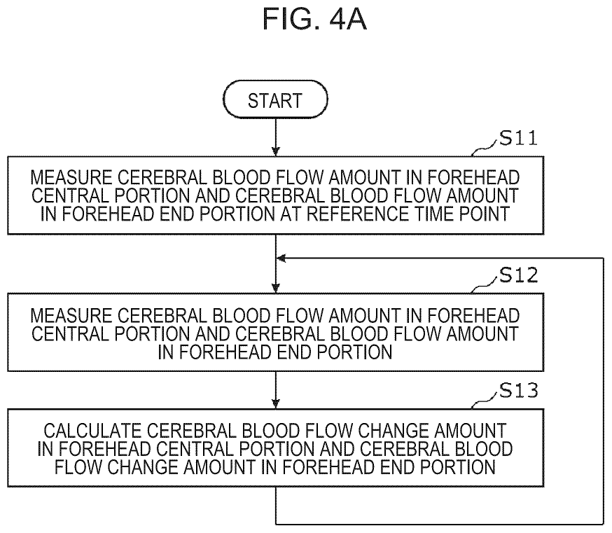

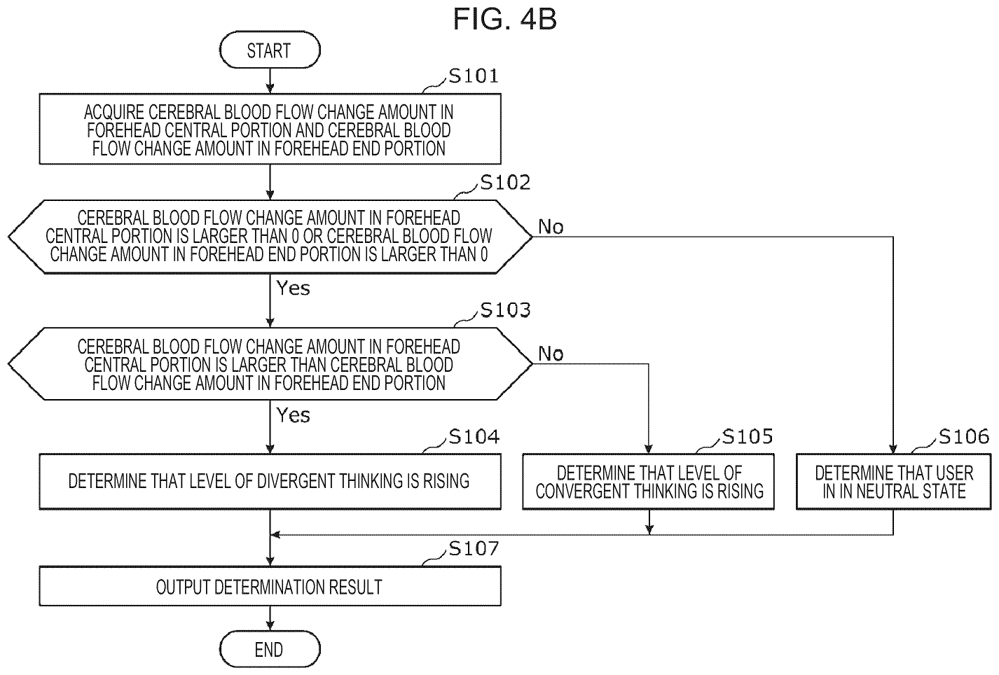

FIG. 4A is a first flowchart illustrating a method for determining the thinking state of the user U performed by the bioinstrumentation device 100 according to the present embodiment. The series of processing illustrated in FIG. 4A is processing for continuously measuring a cerebral blood flow amount in the forehead central portion of the user U and a cerebral blood flow amount in the forehead end portion of the user U.

In step S11, the bioinstrumentation device 100 measures a cerebral blood flow amount in the forehead central portion of the user U and a cerebral blood flow amount in the forehead end portion of the user U at a reference time point by using the camera unit 110 and the processing circuit 134. The processing circuit 134 causes the measured cerebral blood flow amounts of the user U at the reference time point to be stored in the memory 136. The reference time point is a time point during a resting state of the user U and can be, for example, a time point at which the user U starts a task, work, or the like. The reference time point is also referred to as a first time point.

In step S12, the bioinstrumentation device 100 measures a cerebral blood flow amount in the forehead central portion of the user U and a cerebral blood flow amount in the forehead end portion of the user U at a current time point by using the camera unit 110 and the processing circuit 134. The processing circuit 134 causes the measured cerebral blood flow amounts of the user U at the current time point to be stored in the memory 136. The time point at which the process in step S12 is performed is also referred to as a second time point.

In step S13, the bioinstrumentation device 100 calculates change amounts of the cerebral blood flow amount in the forehead central portion of the user U and the cerebral blood flow amount in the forehead end portion of the user U measured in step S12 from the cerebral blood flow amount in the forehead central portion of the user U and the cerebral blood flow amount in the forehead end portion of the user U at the reference time point measured in step S11 by the following formula 1 and formula 2. Note that it can be said that a cerebral blood flow change amount larger than 0 is an increase amount of cerebral blood flow.
the cerebral blood flow change amount in the forehead central portion=the cerebral blood flow amount in the forehead central portion measured in step S12-the cerebral blood flow amount in the forehead central portion at the reference time point (formula 1)
the cerebral blood flow change amount in the forehead end portion=the cerebral blood flow amount in the forehead end portion measured in step S12-the cerebral blood flow amount in the forehead end portion at the reference time point (formula 2)

When the process in step S13 ends, the bioinstrumentation device 100 performs the process in step S12 again. The bioinstrumentation device 100 ends execution of step S12 in a case where determination of the thinking state of the user U is ended.

The bioinstrumentation device 100 performs the processing illustrated in FIG. 4B in parallel while repeatedly performing steps S12 and S13. For example, the bioinstrumentation device 100 performs the processing illustrated in FIG. 4B every time step S13 is performed.

FIG. 4B is a second flowchart illustrating a method for determining the thinking state of the user U performed by the bioinstrumentation device 100 according to the present embodiment.

In step S101, the processing circuit 134 acquires a cerebral blood flow change amount in the forehead central portion of the user U and a cerebral blood flow change amount in the forehead end portion of the user U. The cerebral blood flow change amounts acquired by the camera unit 110 are the cerebral blood flow change amounts of the user U calculated in step S13 (see FIG. 4A).

In step S102, the processing circuit 134 determines whether or not the cerebral blood flow change amount in the forehead central portion acquired in step S101 is larger than 0 or whether or not the cerebral blood flow change amount in the forehead end portion is larger than 0. In a case where the cerebral blood flow change amount in the forehead central portion is larger than 0 or the cerebral blood flow change amount in the forehead end portion is larger than 0 (Yes in step S102), step S103 is performed. Otherwise (No in step S102), step S106 is performed.

In step S103, the processing circuit 134 determines whether or not the cerebral blood flow change amount in the forehead central portion acquired in step S101 is larger than the cerebral blood flow change amount in the forehead end portion. In a case where it is determined that the cerebral blood flow change amount in the forehead central portion is larger than the cerebral blood flow change amount in the forehead end portion (Yes in step S103), step S104 is performed. Otherwise (No in step S103), step S105 is performed.

In step S104, the processing circuit 134 determines that the thinking state of the user U is a state where a level of divergent thinking is rising.

In step S105, the processing circuit 134 determines that the thinking state of the user U is a state where a level of convergent thinking is rising.

In step S106, the processing circuit 134 determines that the thinking state of the user U at the current time point is a state where neither the level of divergent thinking nor the level of convergent thinking is rising (also referred to as a neutral state).

As illustrated in the flowchart of FIG. 4B, in a case where the processing circuit 134 determines in step S106 that the thinking state of the user U at the current time point is a state where neither the level of divergent thinking nor the level of convergent thinking is rising (also referred to as a neutral state), the processing circuit 134 does not perform the comparison between the cerebral blood flow change amount in the forehead central portion and the cerebral blood flow change amount in the forehead end portion illustrated in S103 until the determination in step S106 is performed after the cerebral blood flow change amount in the forehead central portion of the user U and the cerebral blood flow change amount in the forehead end portion of the user U are acquired in step S101. The processing circuit 134 can thus determine the thinking state of the user U while reducing a computation amount.

In step S107, the processing circuit 134 outputs a result of the determination in step S104, S105, or S106 by using the display device 200.

By the processing illustrated in FIGS. 4A and 4B, the bioinstrumentation device 100 can determine whether or not the user U's level of divergent thinking is rising or whether or not the user U's level of convergent thinking is rising as determination of the thinking state of the user U.

Note that the processing circuit 134 can determine that the user U's level of divergent thinking is rising by performing the following processing instead of the processing illustrated in FIGS. 4A and 4B or together with the processing illustrated in FIGS. 4A and 4B.

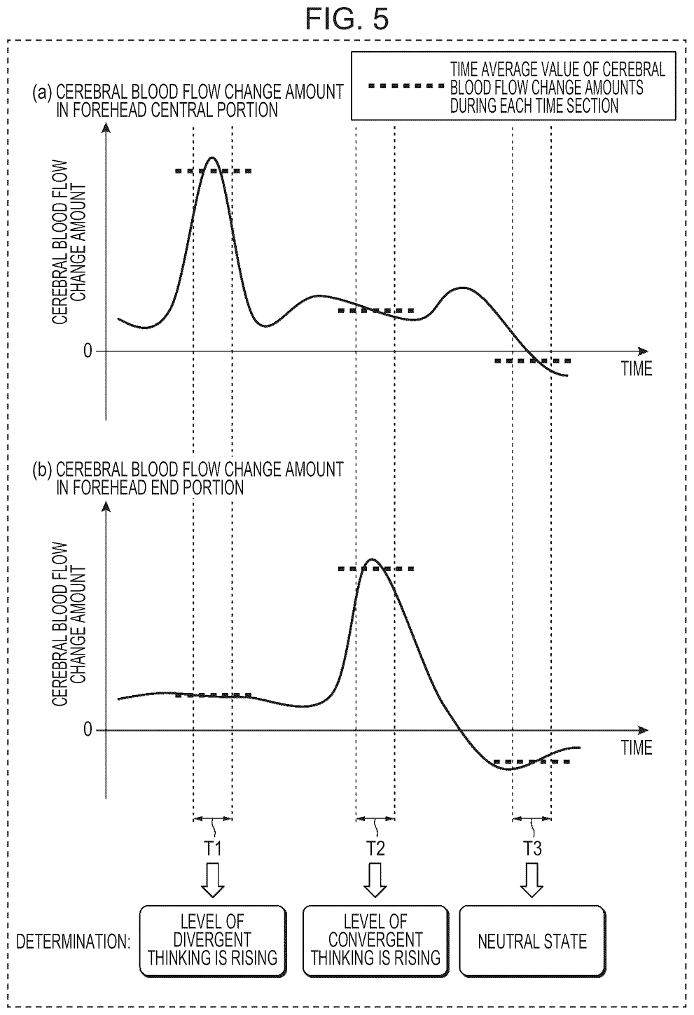

That is, the processing circuit 134 can determine that the user U's level of divergent thinking is rising in a case where a rate of change of the cerebral blood flow amount in the forehead central portion per time is higher than a rate of change of the cerebral blood flow amount in the forehead end portion per time. In this case, it is assumed that the first sensor is repeatedly acquiring the cerebral blood flow amount in the forehead central portion and the second sensor is repeatedly acquiring the cerebral blood flow amount in the forehead end portion. Here, the rate of change of a cerebral blood flow amount per time corresponds to a slope of a graph (see FIG. 5) illustrating a temporal change of cerebral blood flow, in other words, corresponds to a differential value of the temporal change of the cerebral blood flow.

FIG. 5 is an explanatory view illustrating an example of the user U's thinking state determined by the bioinstrumentation device 100 according to the present embodiment.

FIG. 5(a) illustrates a temporal change of a cerebral blood flow change amount in the forehead central portion of the user U over time, and FIG. 5(b) illustrates a temporal change of a cerebral blood flow change amount in the forehead end portion of the user U over time.

The following describes time sections T1, T2, and T3 illustrated in FIG. 5.

In the case of the time section T1, the processing circuit 134 determines in step S102 that the cerebral blood flow change amount in the forehead central portion is larger than 0 and the cerebral blood flow change amount in the forehead end portion is larger than 0. Furthermore, the processing circuit 134 determines in step S103 that the cerebral blood flow change amount in the forehead central portion is larger than the cerebral blood flow change amount in the forehead end portion. As a result, the processing circuit 134 determines that the thinking state of the user U is a state where the level of divergent thinking is rising (step S104).

In the case of the time section T2, the processing circuit 134 determines in step S102 that the cerebral blood flow change amount in the forehead central portion is larger than 0 and the cerebral blood flow change amount in the forehead end portion is larger than 0. Furthermore, the processing circuit 134 determines in step S103 that the cerebral blood flow change amount in the forehead central portion is not larger than the cerebral blood flow change amount in the forehead end portion. As a result, the processing circuit 134 determines that the thinking state of the user U is a state where the level of convergent thinking is rising (step S105).

In the case of the time section T3, the processing circuit 134 determines in step S102 that the cerebral blood flow change amount in the forehead central portion is not larger than 0 and the cerebral blood flow change amount in the forehead end portion is not larger than 0. As a result, the processing circuit 134 determines that the thinking state of the user U is a neutral state (step S106).

Display of information concerning a state of the user U is described below. Presented information includes a determination result output by the processing circuit 134 in step S107 of FIG. 4B and is presented to the user U by using the display device 200.

The display device 200 presents the thinking state of the user U determined by the bioinstrumentation device 100, in other words, visualizes the thinking state of the user U (makes the thinking state of the user U visible).

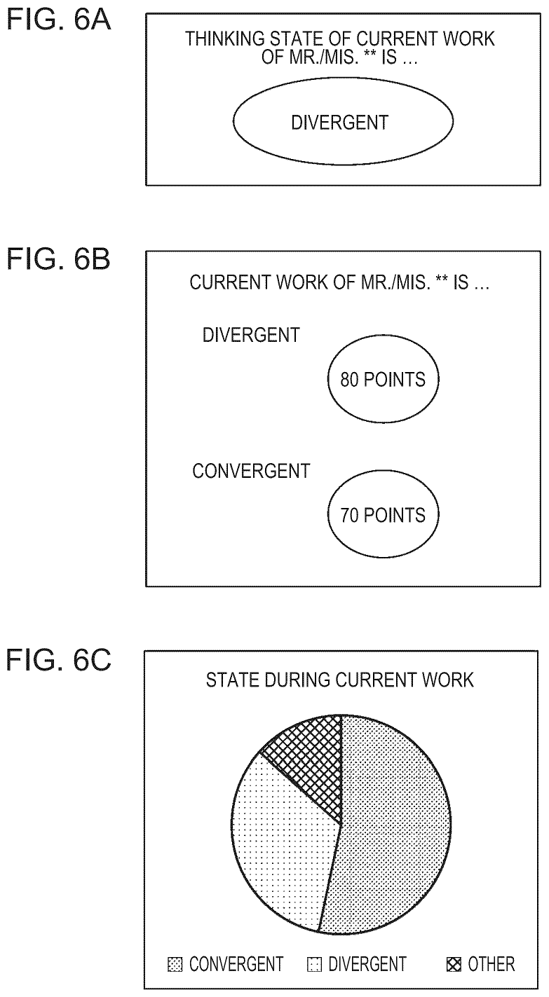

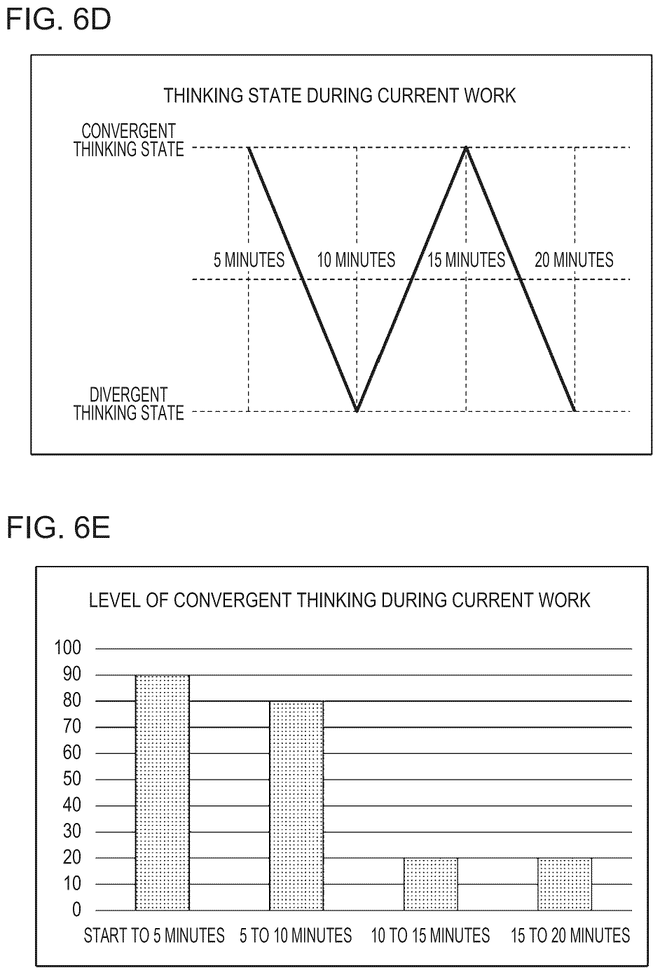

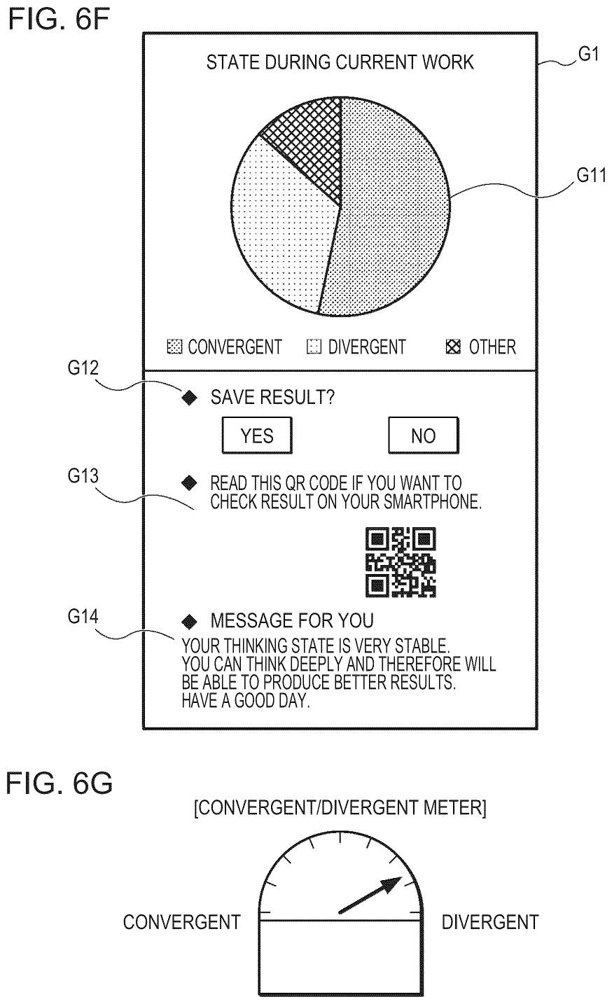

A diagram (e.g., a graph) illustrating a determination result presented by the display device 200 may be one illustrating the thinking state of the user U (i.e., whether or not the level of convergent thinking is rising or whether or not the level of divergent thinking is rising) in a time-series manner (FIG. 6D) or may be one illustrating proportions or a ratio of the thinking state of the user U (FIG. 6C or 6E). Furthermore, only the thinking state of the user U may be displayed (FIG. 6A, 6C, or 6D) or not only the thinking state of the user U, but also an evaluation value of determination of the thinking state may be displayed (FIG. 6B or 6E). The evaluation value may be a value indicative of validity as the thinking state of the user U, may be a value indicative of stability of thinking based on a period for which a certain thinking state has been continuously maintained, or may be a value indicative of a proportion of a period of a certain thinking state in a task period. In a case where the evaluation value is displayed, a score such as “convergent thinking x points” or “divergent thinking y points” may be displayed (FIG. 6B). The convergent thinking may be expressed by a more general expression “concentrated”, and the divergent thinking may be expressed by a more general expression “creative”. The determination result may be a result of evaluation and determination of whole working hours or may be a result of determination of a state in a specific time zone.

In a case where the processing circuit 134 determines the thinking state of the user U and then scores the determined thinking state, the processing circuit 134 may decide the score on the basis of a change amount from biological information in a resting state of the user U acquired in advance to biological information in the performing state. The processing circuit 134 may calculate the score by comparing an average value of biological information measured during the performing state of the user U and a value of an average feature amount of data of persons acquired in advance by an experiment. Furthermore, the processing circuit 134 may calculate the score by comparing a change amount from biological information in the resting state of the user U acquired in advance to biological information in the performing state and a value of an average fluctuation amount of feature amount data of persons acquired in advance by an experiment.

FIGS. 6A to 6G illustrate an example of display of the determination result according to the present embodiment.

FIG. 6A is an explanatory view of a first example of display of the determination result according to the present embodiment. FIG. 6A illustrates an example of displaying tendency of the thinking state of the user U during work as a word. The “DIVERGENT” in FIG. 6A is an abbreviation of “divergent thinking”. An abbreviation of the “convergent thinking” can be “CONVERGENT”. The same applies hereinafter. Note that the display of FIG. 6A is not limited to display on a screen and may be audio presentation.

FIG. 6B is an explanatory view of a second example of display of the determination result according to the present embodiment. FIG. 6B illustrates an example of displaying a determination result of the thinking state of the user U during work as a score. The displayed score may be only a score concerning a specific thinking state or may be scores concerning thinking states. An upper limit value of the score may be 100 or may be 5, for example.

FIG. 6C is an explanatory view of a third example of display of the determination result according to the present embodiment. FIG. 6C illustrates an example of showing a proportion of a thinking state (i.e., convergent thinking or divergent thinking) of the user U during work as a pie chart.

FIG. 6D is an explanatory view of a fourth example of display of the determination result according to the present embodiment. FIG. 6D illustrates an example of showing a thinking state of the user U during work in a time-series manner. The horizontal axis represents a time, and the vertical axis represents the thinking state (i.e., convergent thinking or divergent thinking) of the user U. Note that the time represented by the horizontal axis may indicate one time point or may indicate a period of several minutes.

FIG. 6E is an explanatory view of a fifth example of display of the determination result according to the present embodiment. FIG. 6E illustrates an example of showing a score (also referred to as a convergent thinking level) of convergent thinking of the user U during work in a time-series manner. The horizontal axis represents a time, and the vertical axis represents a score of convergent thinking of the user U. Note that the vertical axis may represent a score of divergent thinking. Results of thinking states may be displayed in a unified manner.

FIG. 6F is an explanatory view of a sixth example of presentation of the determination result according to the present embodiment. FIG. 6F illustrates a presentation example including information other than a determination result. FIG. 6F illustrates, as presented information, an example of a display image G1 displayed on the display device 200. The display image G1 includes a graph G11, an input field G12, and message display fields G13 and G14.

The graph G11 is similar to the graph illustrated in FIG. 6C.

The input field G12 is a field where the user U selects and inputs whether to save a determination result. In the input field G12, a message “SAVE RESULT?”, a “YES” button, and a “NO” button are displayed. The “YES” button is a button by which the user U selects to save a determination result. The “NO” button is a button by which the user U selects not to save a determination result. When the user U performs operation of selecting the “YES” button, the display device 200 receives the operation, and changes a color of the “YES” button. When the user U performs operation of selecting the “NO” button, the display device 200 receives the operation, and changes a color of the “NO” button. This allows the user U to easily confirm which button has been selected.

When the user U performs operation of selecting the “YES” button, the display device 200 receives the operation, and as a result, the determination result is “permitted” to be saved, and the determination result concerning the thinking state of the user U is saved. When the user U performs operation of selecting the “NO” button, the display device 200 receives the operation, and as a result, the determination result is “not permitted” to be saved, and the determination result concerning the thinking state of the user U is not saved. Since the determination result that is not permitted is not saved in the storage unit, it is possible to reduce unnecessary consumption of the memory.

The message display field G13 includes a QR code (Registered Trademark). The message display field G13 also includes a message notifying the user U that the user U can check the result on his or her mobile terminal by reading the QR code. In FIG. 6F, the message display field G13 includes a message “READ THIS QR CODE IF YOU WANT TO CHECK RESULT ON YOUR SMARTPHONE” and a QR code. Although a QR code is illustrated in this example, an e-mail address of the user U may be entered.

Alternatively, the display device 200 may be equipped with a card reader, and when a card such as an employee ID card is read by the card reader, a determination result may be transmitted to the user U and his or her superior.

The message display field G14 includes a message taking into consideration user U's biological information during work, the weather, current time, and the like. In FIG. 6F, a positive message “YOUR THINKING STATE IS VERY STABLE. YOU CAN THINK DEEPLY AND THEREFORE WILL BE ABLE TO PRODUCE BETTER RESULTS. HAVE A GOOD DAY.” is displayed assuming that the user U maintained a specific thinking state for a long period in early morning.

FIG. 6G is a seventh explanatory view of display of the determination result according to the present embodiment.

FIG. 6G illustrates an example of showing a proportion of a thinking state (i.e., convergent thinking or divergent thinking) of the user U during work in a form of a meter. Proportions of convergent thinking and divergent thinking are indicated by a position indicated by the arrow.

In the present embodiment, the bioinstrumentation device 100 may control a lighting device to change a color temperature or illuminance of illuminating light that illuminates an area around the user U in accordance with a determination result. For example, the user U may input, to the bioinstrumentation device 100 in advance, information indicating that the user U wants to perform a task in a certain thinking state, and in a case where the task starts in a certain lighting state, illuminance or a color temperature of a room may be changed at certain time intervals during the task in accordance with a determination result of the thinking state of the user U. Furthermore, in a case where the user U feels lighting in a certain period comfortable, the user U may be allowed to save the lighting setting by using an operation terminal connected to the bioinstrumentation device 100. In a case where the user U wants to be in the same thinking state next time, a task may be started by using the saved lighting setting.

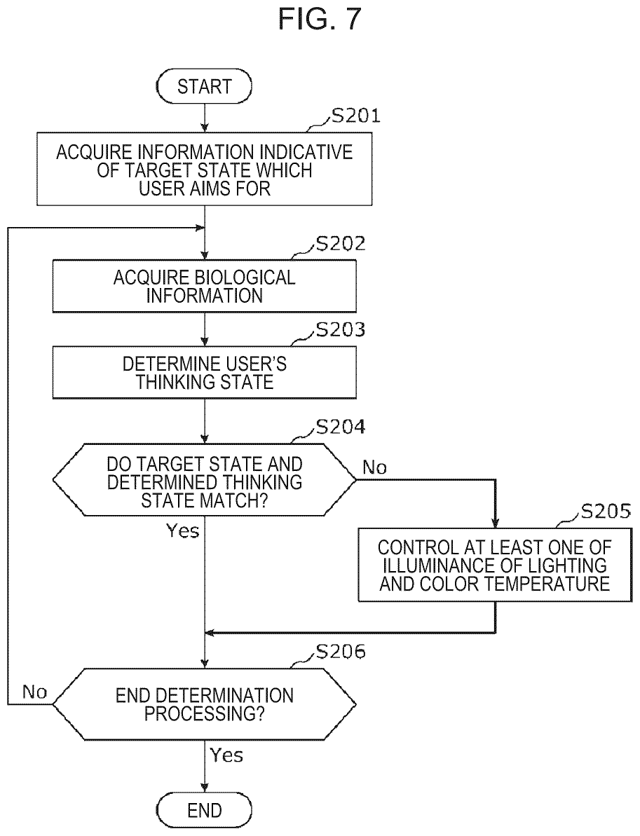

FIG. 7 is a flowchart illustrating an example of processing for controlling lighting in accordance with a determination result according to the present embodiment.

In step S201, the bioinstrumentation device 100 acquires information concerning a target thinking state which the user U aims for. The information is acquired, for example, when user U's input of information is received by using an input device such as a keyboard, a mouse, a touch pad, or a microphone.

The processes in step S202 and step S203 are similar to the processes in step S101 to step S107 in FIG. 4B.

In step S204, the processing circuit 134 determines whether or not the target thinking state acquired in step S201 and the thinking state determined in step S203 match. Specifically, the processing circuit 134 determines that these thinking states match in a case where the target thinking state is a divergent thinking state and it is determined that a level of divergent thinking is rising. Furthermore, the processing circuit 134 determines that these thinking states match in a case where the target thinking state is a convergent thinking state and it is determined that a level of convergent thinking is rising. In the other cases, the processing circuit 134 determines that these thinking states do not match. In a case where it is determined that these thinking states match (Yes in step S204), step S206 is performed. Otherwise (No in step S204), step S205 is performed.

In step S205, the processing circuit 134 controls at least one of illuminance or a color temperature of lighting. Details of the control will be described later.

In step S206, the processing circuit 134 determines whether or not to end the series of determination processing illustrated in FIG. 7. For example, the processing circuit 134 may determine whether or not acquisition of new biological information (step S202) succeeds, and may determines that the series of determination processing illustrated in FIG. 7 is ended in a case where it is determined that the acquisition of new biological information does not succeed. The processing circuit 134 may determine whether or not a preset period has elapsed, and may determines that the series of determination processing illustrated in FIG. 7 is ended in a case where it is determined that the period has elapsed. By ending the determination processing, a processing amount of the processor can be reduced.

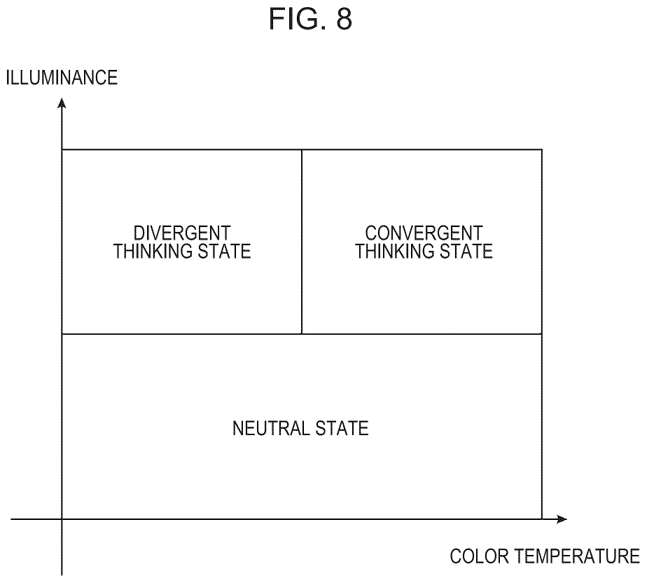

FIG. 8 illustrates a relationship between illuminance and a color temperature of lighting suitable for guiding the user U to a certain thinking state (i.e., convergent thinking or divergent thinking). Such a relationship between illuminance and a color temperature of lighting is described in Koichiro FUMOTO, Yutaka HASHIURA, Kouzou TSUJI, Takeki KIMURA, “Office lighting system to encourage creativity of workers-Study on optimal lighting conditions for creative work in the office-”, Japan Human Factors and Ergonomics Society KANSAI Branch conference proceedings 2009, pages 171 to 174, Dec. 5, 2009 (hereinafter referred to as Non Patent Literature 1).

Therefore, the bioinstrumentation device 100 can guide the user U to a divergent thinking state or a convergent thinking state, for example, by controlling the lighting device. The bioinstrumentation device 100 can control the lighting device, for example, by transmitting, to the lighting device, communication data including a control command to change a color temperature or illuminance of illuminating light to be emitted by the lighting device. Upon receipt of the communication data, the lighting device changes a color temperature or illuminance of illuminating light to be emitted in accordance with the control command included in the received communication data.

(1) Control for guising the user U to a divergent thinking state
For example, Non Patent Literature 1 describes using high-illuminance and low-color-temperature lighting as a condition suitable for work such as brainstorming, that is, as a condition suitable for a divergent thinking state.

In a case where the user U is to be guided to a divergent thinking state, it is estimated that the thinking state of the user U is not a state where a level of divergent thinking is rising, but is, for example, a state where a level of convergent thinking is rising or a neutral state.

In this case, there is a possibility that the bioinstrumentation device 100 can guide the user U to a divergent thinking state by controlling the lighting device to lower a color temperature of illuminating light and raise illuminance of the illuminating light (see FIG. 8).

Therefore, there is a possibility that the bioinstrumentation device 100 can guide the user U to a divergent thinking state by controlling the lighting device to lower a color temperature of illuminating light and raise illuminance of the illuminating light in a case where it is determined that the cerebral blood flow change amount in the forehead central portion of the user U is larger than 0 or the cerebral blood flow change amount in the forehead end portion is larger than 0 and it is determined that the cerebral blood flow change amount in the forehead central portion from the reference time point to the current time point is not larger than the cerebral blood flow change amount in the forehead end portion from the reference time point to the current time point (i.e., in a case where it is determined that a level of convergent thinking is rising).

Furthermore, there is a possibility that the bioinstrumentation device 100 can guide the user U to a divergent thinking state by controlling the lighting device to lower a color temperature of illuminating light and raise illuminance of the illuminating light in a case where it is determined that both of the cerebral blood flow change amount in the forehead central portion and the cerebral blood flow change amount in the forehead end portion are smaller than 0 (i.e., in a case where it is determined that the user U is in a neutral state).

(2) Control for guiding the user U to a convergent thinking state
For example, Non Patent Literature 1 describes using high-color-temperature and high-illuminance lighting as a condition suitable for a convergent thinking state.

In a case where the user U is to be guided to a convergent thinking state, it is estimated that the thinking state of the user U is not a state where a level of convergent thinking is rising, but is, for example, a state where a level of divergent thinking is rising or a neutral state.

In this case, there is a possibility that the bioinstrumentation device 100 can guide the user U to a convergent thinking state by controlling the lighting device to raise a color temperature of illuminating light and raise illuminance of the illuminating light (see FIG. 8).

Therefore, there is a possibility that the bioinstrumentation device 100 can guide the user U to a convergent thinking state by controlling the lighting device to raise a color temperature of illuminating light and raise illuminance of the illuminating light in a case where it is determined that the cerebral blood flow change amount in the forehead central portion of the user U is larger than 0 or the cerebral blood flow change amount in the forehead end portion is larger than 0 and it is determined that the cerebral blood flow change amount in the forehead central portion from the reference time point to the current time point is larger than the cerebral blood flow change amount in the forehead end portion from the reference time point to the current time point (i.e., in a case where it is determined that a level of convergent thinking is rising).

Furthermore, there is a possibility that the bioinstrumentation device 100 can guide the user U to a convergent thinking state by controlling the lighting device to raise a color temperature of illuminating light and raise illuminance of the illuminating light in a case where it is determined that both of the cerebral blood flow change amount in the forehead central portion and the cerebral blood flow change amount in the forehead end portion are smaller than 0 (i.e., in a case where it is determined that the user U is in a neutral state).

As described above, the bioinstrumentation device 100 makes it possible to objectively evaluate a subject's creative thinking state.

In the above embodiment, each constituent element may be realized by dedicated hardware or may be realized by execution of a software program suitable for the constituent element. Each constituent element may be realized by a program executing unit such as a CPU or a processor reading out and executing a software program stored in a recording medium such as a hard disk or a semiconductor memory. Software realizing the information processing device and others according to the above embodiment is the following program.

Specifically, this program causes a computer to execute an information processing method including acquiring first cerebral blood flow information corresponding to a temporal change of cerebral blood flow in a central portion of a user's forehead in a left-right direction by using a sensor, acquiring second cerebral blood flow information corresponding to a temporal change of cerebral blood flow in an end portion of the user's forehead in the left-right direction by using the sensor, and determining by a processor whether a user's level of divergent thinking is rising or a user's level of convergent thinking is rising on the basis of comparison between the first cerebral blood flow information and the second cerebral blood flow information.

Any of the CPU and the processor that perform the thinking state determination processing and the storage device may be included in a server that communicates with the sensor that detects user's biological information over a network.

The information processing method and others according to one or more aspects have been described above on the basis of the embodiment, but the present disclosure is not limited to this embodiment. Various modifications of the present embodiment which a person skilled in the art can think of and combinations of constituent elements in different embodiments are also encompassed within the scope of the one or more aspects without departing from the spirit of the present disclosure.

The present disclosure is applicable to a device that senses a human state.

What is claimed is:

1. An information processing method comprising:
acquiring first cerebral blood flow information corresponding to a temporal change of cerebral blood flow in a central portion of a user's forehead in a left-right direction by using a sensor;
acquiring second cerebral blood flow information corresponding to a temporal change of cerebral blood flow in an end portion of the user's forehead in the left-right direction by using the sensor; and
determining by a processor whether a user's level of divergent thinking is rising or a user's level of convergent thinking is rising on a basis of comparison between the first cerebral blood flow information and the second cerebral blood flow information.

2. The information processing method according to claim 1, wherein
the sensor acquires a cerebral blood flow amount in the central portion at a first time point and a cerebral blood flow amount in the central portion at a second time point later than the first time point and acquires a cerebral blood flow amount in the end portion at the first time point and a cerebral blood flow amount in the end portion at the second time point, and
the determining includes determining that the user's level of divergent thinking is rising in a case where it is determined that an increase amount of the cerebral blood flow amount in the central portion at the second time point from the cerebral blood flow amount in the central portion at the first time point is larger than an increase amount of the cerebral blood flow amount in the end portion at the second time point from the cerebral blood flow amount in the end portion at the first time point.

3. The information processing method according to claim 1, wherein
the sensor repeatedly acquires a cerebral blood flow amount in the central portion and repeatedly acquires a cerebral blood flow amount in the end portion, and
the determining includes determining that the user's level of divergent thinking is rising in a case where it is determined that a rate of change of the cerebral blood flow amount in the central portion per time is higher than a rate of change of the cerebral blood flow amount in the end portion per time.

4. The information processing method according to claim 1, wherein
the sensor acquires a cerebral blood flow amount in the central portion at a first time point and a cerebral blood flow amount in the central portion at a second time point later than the first time point and acquires a cerebral blood flow amount in the end portion at the first time point and a cerebral blood flow amount in the end portion at the second time point, and
the determining includes determining that the user's level of convergent thinking is rising in a case where it is determined that an increase amount of the cerebral blood flow amount in the end portion at the second time point from the cerebral blood flow amount in the end portion at the first time point is larger than an increase amount of the cerebral blood flow amount in the central portion at the second time point from the cerebral blood flow amount in the central portion at the first time point.

5. The information processing method according to claim 1, wherein
the sensor acquires the first cerebral blood flow information and the second cerebral blood flow information in each of periods,
the determining includes determining, for each of the periods, whether a user's level of divergent thinking in the period is rising or a user's level of convergent thinking in the period is rising on a basis of the first cerebral blood flow information and the second cerebral blood flow information acquired in the period, and
the information processing method further comprises generating a graph showing proportions of a period where the user's level of convergent thinking is rising and a period where the user's level of divergent thinking is rising among the periods and displaying the graph.

6. The information processing method according to claim 2, further comprising controlling a lighting device that illuminates an area around the user to raise a color temperature of illuminating light and raise illuminance of the illuminating light.

7. The information processing method according to claim 4, further comprising controlling a lighting device that illuminates an area around the user to lower a color temperature of illuminating light and raise illuminance of the illuminating light.

8. An information processing method comprising:
causing a processor to:
acquire target information indicative of a target state which a user aims for;
acquire cerebral blood flow information indicative of a temporal change of cerebral blood flow of the user;
determine a thinking state of the user on a basis of the cerebral blood flow information;
compare the target state indicated by the target information and the determined thinking state of the user; and
perform control of changing a property of illuminating light emitted by a lighting device that illuminates an area around the user in a case where the target state and the determined thinking state of the user do not match as a result of the comparison.

9. An information processing system comprising:
a light source that emits first light to a central portion of a user's forehead in a left-right direction and emits second light to an end portion of the user's forehead in the left-right direction;
a detector that detects third light originating from the first light and coming from the central portion and fourth light originating from the second light and coming from the end portion;
a processing circuit; and
a display device, wherein
the processing circuit
generates first cerebral blood flow information including information on a temporal change of a hemoglobin concentration of cerebral blood flow in the central portion on a basis of the third light,
generates second cerebral blood flow information including information on a temporal change of a hemoglobin concentration of cerebral blood flow in the end portion on a basis of the fourth light,
performs comparison processing of comparing a change amount of the hemoglobin concentration of the cerebral blood flow in the central portion and a change amount of the hemoglobin concentration of the cerebral blood flow in the end portion on a basis of the first cerebral blood flow information and the second cerebral blood flow information,
performs determination processing of determining that a user's level of divergent thinking is rising in a case where it is determined on a basis of the comparison processing that the change amount of the hemoglobin concentration of the cerebral blood flow in the central portion is larger than the change amount of the hemoglobin concentration of the cerebral blood flow in the end portion, and determining that a user's level of convergent thinking is rising in a case where it is determined on a basis of the comparison processing that the change amount of the hemoglobin concentration of the cerebral blood flow in the end portion is larger than the change amount of the hemoglobin concentration of the cerebral blood flow in the central portion, and
displays a result of the determination processing on a display.
